(19) United States
(12) Patent Application Publication
(10) Pub. No.: US 2021/0038777 A1
(43) Pub. Date: Feb. 11, 2021

(54) VISUAL BEAUTY INSTRUMENT THAT SUPPORTS SKIN BLACKHEAD ANALYSIS

(71) Applicant: POWER 7 TECHNOLOGY CORP. (Shenzhen), Shenzhen (CN)

(72) Inventors: Mike CHEN, Shenzhen (CN); Yan-Qiu ZHANG, Shenzhen (CN)

(21) Appl. No.: 16/566,678

(22) Filed: Sep. 10, 2019

Related U.S. Application Data
(63) Continuation-in-part of application No. 16/531,386, filed on Aug. 5, 2019.

(51) Int. Cl. A61M 1/00; B08B 5/04; H04N 5/225; G06K 9/00

(52) U.S. Cl. CPC A61M 1/0031; B08B 5/04; A61M 1/0049; H04N 5/2256; H04N 5/2253; G06K 9/00624; A61B 5/0077; H04N 5/2257; A61M 2205/502; A61M 2205/583; A61M 2205/3306; A61M 2210/04; H04N 5/22525

(57) ABSTRACT

A visual beauty instrument includes a control module, an image acquisition module, a suction control module, a fill light module, a suction nozzle assembly and a mobile terminal. The image acquisition module collects the image signal of the suction area and feeds back the image signal to the control module. The control module analyzes the image signal and controls the suction control module and the fill light module. The suction control module sucks up the acne and blackheads on the skin surface by the suction nozzle assembly. The fill light module improves the recognition of acne and blackheads. The invention can observe the distribution and size of blackheads and acne in the inner layer of the skin and compare the number of blackheads and acne before and after suction, ensuring thorough cleaning, improving removal efficiency and preventing skin damage caused by excessive suction.

This application is a Continuation-In-Part of co-pending application Ser. No. 16/531,386, filed on Aug. 5, 2019, for which priority is claimed under 35 U.S.C. § 120, the entire contents of which are hereby incorporated by reference.

### 1. FIELD OF THE INVENTION

The present invention relates to beauty and video image processing and more particularly, to a visual beauty instrument that supports skin blackhead analysis.

### 2. DESCRIPTION OF THE RELATED ART

Blackhead is a “plug” of sebum, cell debris and bacteria commonly seen on the skin (especially on the face), which plagues most people. At present, the methods for removing blackheads are chemical mask adsorption and instrument suction. The use of chemical mask adsorption to remove blackheads has a poor adsorption effect. Long-term use of chemical masks containing various chemical components can cause damage to the skin.

The general industry skin beauty instrument includes a charging management circuit, a battery protection circuit, an MCU main control circuit, a constant voltage control circuit, a beauty lamp control circuit and a beauty lamp circuit. The charging management circuit is electrically connected with the battery protection circuit. The MCU main control circuit is respectively electrically connected to the battery protection circuit, the constant voltage control circuit and the beauty lamp control circuit. The lamp control circuit is electrically connected to the beauty lamp circuit. The battery protection circuits respectively electrically connected to the constant voltage control circuit and the beauty lamp control circuit.

The above design stabilizes the output to a certain extent, but it still has at least the following defects: First, during the process of blackhead removing, it is impossible to observe the surface of the skin, affecting the blackhead removal effect. Second, it is impossible to observe the distribution and size of blackheads and acne in the inner layer of the skin. Third, it is impossible to compare the number of blackheads and acne before and after suction.

### SUMMARY OF THE INVENTION

The present invention has been accomplished under the circumstances in view. It is therefore the main object of the present invention to provide a visual beauty instrument, which can observe the distribution and size of blackheads and acne in the inner layer of the skin, and compare the number of blackheads and acne before and after suction, ensuring thorough cleaning, improving removal efficiency and preventing skin damage caused by excessive suction.

To achieve this and other objects of the present invention, a visual beauty instrument comprises a control module, an image acquisition module, a suction control module, a fill light module, a suction nozzle assembly and a mobile terminal, wherein the image acquisition module collects the image signal of the suction area and feeds back the image signal to the control module; the control module analyzes the image signal and controls the suction control module and the fill light module; the suction control module sucks up the acne and blackheads on the skin surface by the suction nozzle assembly; the fill light module improves the recognition of acne and blackheads.

Preferably, the visual beauty instrument further comprises a mobile terminal. The mobile terminal is communicatively connected with the control module. The mobile terminal is integrated with an image analysis module and an image display module. The image analysis module is used to analyze image information.

Preferably, the suction nozzle assembly comprises a nozzle body, a nozzle tip capped on the nozzle body, and a light-transmitting lens and a sealing ring disposed between the nozzle body and the nozzle tip. Further, the nozzle tip has a silicone portion disposed at a suction opening thereof.

Preferably, the suction control module comprises a vacuum pump, and a motor that controls the vacuum pump. The vacuum pump has an air inlet hole and an air outlet hole. The air outlet hole communicates with the external space. The air inlet hole communicates with the nozzle body by a tube.

Preferably, the visual beauty instrument further comprises a power module. The power module comprises a battery, and a battery management device electrically connected with the battery. The battery management device is respectively electrically connected to the control module, the image acquisition module, the suction control module, the fill light module and the mobile terminal.

Preferably, the visual beauty instrument further comprises a housing, and a control button set mounted on the housing. The control button set comprises a power button and a mode switching button. The power button and the mode switching button are respectively electrically connected to the control module.

Preferably, the visual beauty instrument further comprises an indicator light set. The indicator light set comprises a plurality of suction force level indicator lights and a power status indicator light. The suction force level indicator lights and the power status indicator light are electrically connected to the control module.

In general, the visual beauty instrument of the present invention comprises a control module, an image acquisition module, a suction control module, a fill light module, a suction nozzle assembly and a mobile terminal. The image acquisition module collects the image signal of the suction area and feeds back the image signal to the control module. The control module analyzes the image signal and controls the suction control module and the fill light module. The suction control module sucks up the acne and blackheads on the skin surface by the suction nozzle assembly. The fill light module improves the recognition of acne and blackheads. The image acquisition module is a fixed-focus macro camera with multiple image enlargement function and digital zoom function. It is used to capture the image or video of the skin in the suction area and send the captured image or video to the control module for analyzing the distribution and size of acne and blackheads in the inner layer. Then, the control module controls the suction control module to output the negative pressure of different levels to adjust the suction, avoiding redness or damage to the skin due to excessive suction. At the same time, during image acquisition, the control module controls the fill light module to fill light, improving image quality the recognition of acne and blackheads. When the suction nozzle assembly is in contact with the skin, the vacuum pressure generated by the suction control module sucks and removes the acne and blackheads deep in the skin. The invention can observe the distribution and size of blackheads and acne in the inner layer of the skin and compare the number of blackheads and acne before and after suction, ensuring thorough cleaning, improving removal efficiency and preventing skin damage caused by excessive suction.

### BRIEF DESCRIPTION OF THE DRAWINGS

FIG. 1 is an elevational view of a visual beauty instrument in accordance with the present invention.

FIG. 2 is an exploded view of the suction nozzle assembly.

FIG. 3 is a circuit block diagram of the present invention.

### DETAILED DESCRIPTION OF THE PREFERRED EMBODIMENT

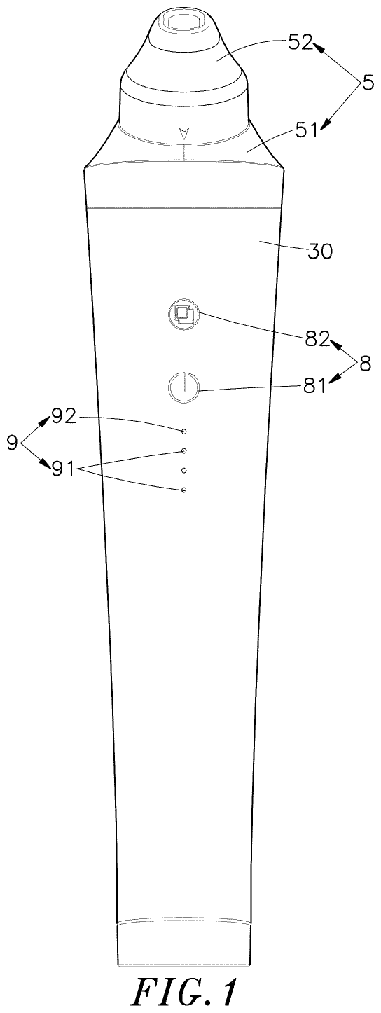

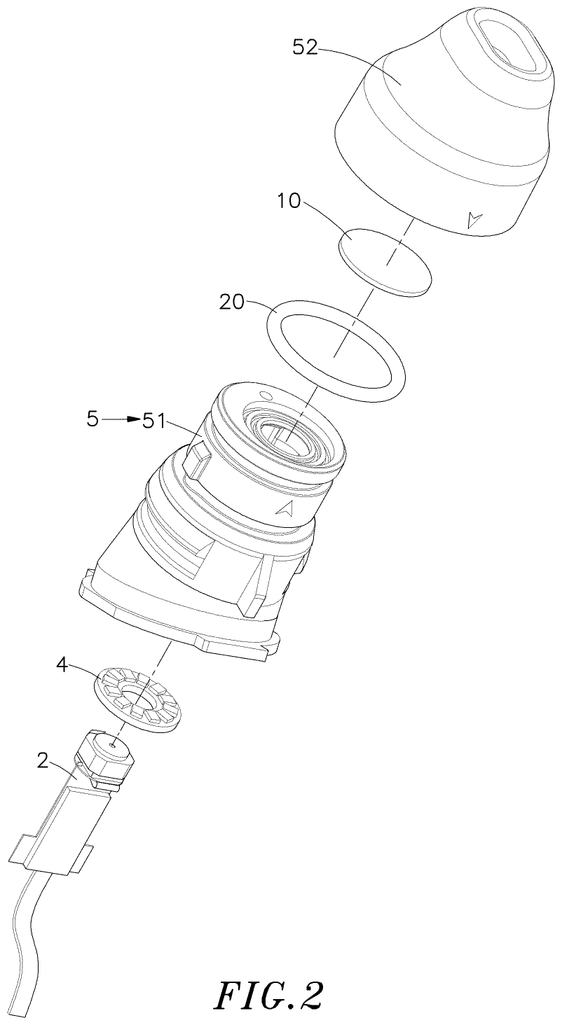

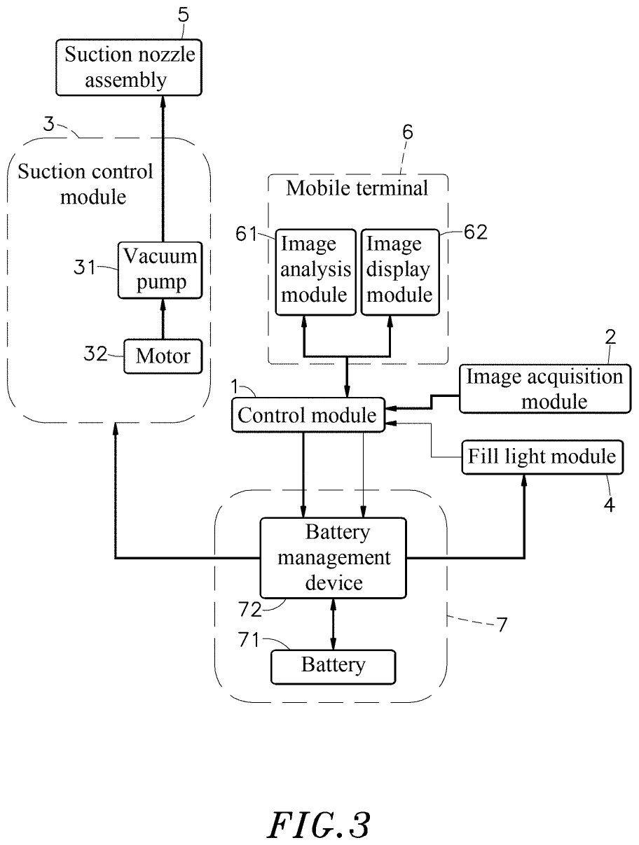

Referring to FIGS. 1-3, a visual beauty instrument that supports skin blackhead analysis generally comprises a control module 1, an image acquisition module 2, a suction control module 3, a fill light module 4 and a suction nozzle assembly 5.

The image acquisition module 2 is used to collect the image signal of the suction area and feed back to the control module 1. The control module 1 analyzes the image signal and controls the suction control module 3 and the fill light module 4. The suction control module 3 sucks up the acne and blackheads on the skin surface by the suction nozzle assembly 5. The fill light module 4 is used to improve the recognition of acne and blackheads. The image acquisition module 2 is a fixed-focus macro camera with multiple image enlargement function and digital zoom function. It is used to capture the image or video of the skin in the suction area and send the captured image or video to the control module 1 for analyzing the distribution and size of acne and blackheads in the inner layer. Then, the control module 1 controls the suction control module 3 to output the negative pressure of different levels to adjust the suction, avoiding redness or damage to the skin due to excessive suction. At the same time, during image acquisition, the control module 1 controls the fill light module 4 to fill light, improving image quality the recognition of acne and blackheads. When the suction nozzle assembly 5 is in contact with the skin, the vacuum pressure generated by the suction control module 3 sucks and removes the acne and blackheads deep in the skin. The control module 1 is ARM9 SOC processing chip, model US6808, it supports USB 2.0 high-speed communication and USB OHCI/EHCI host controller and transceiver. The image acquisition module 2 uses a 1 million-pixel sensor camera connected to the US6808 chip of the control module 1 using the USB protocol. It supports 720P HD video and 1280*720 picture. The camera is installed in the middle of the suction nozzle assembly 5 to capture the skin surface and blackheads and acne in the suction area, preventing blind suction and poor cleaning.

Preferably, the visual beauty instrument further comprises a mobile terminal 6. The mobile terminal 6 and the control module 1 are communicatively connected. The mobile terminal 6 is integrated with an image analysis module 61 and an image display module 62. The image analysis module 61 is used to analyze image information. The image display module 62 is used to convert image information into images or videos. The image analysis module 61 and the image display module 62 are integrated on the app of mobile terminal 6 and communicate with the control module 1. The image analysis module 61 has a DSP image computing capability and a bidirectional communication function with the control module 1.

Preferably, the data exchange between the mobile terminal 6 and the control module 1 is a data line connection or a WiFi AP mode, and the connection interface of the data line is a USB-A, USB TYPE-C or MICRO USB interface.

Preferably, the mobile terminal 6 is an iOS system terminal or an Android system terminal. The mobile terminal 6 is equipped with a touch screen. Through the touch screen, you can realize the functions of taking photos and videos, browsing and playing functions of photos and video files, sharing functions of related files, copying, pasting and deleting functions. Further, through instant two-way communication with control module 1, you can display the battery power of the visual beauty instrument on the touch screen of the mobile terminal 6.

Preferably, the fill light module 4 comprises a plurality of sets of LED lights, including one set of white LED lights, one set of UVA LED lights having a wavelength in the range of 360 to 400 nm, one set of blue LED lights having a wavelength in the range of 410 to 420 nm, and one set of red LED lights having a wavelength in the range of 630 to 640 nm. The set of white LED lights is used to fill light during image acquisition to improve image quality. The set of blue LED lights in the range of 410 to 420 nm is used to treat acne and acne anti-inflammatory. The set of UVA LED lights in the range of 360 to 400 nm is used to make a short fill light when the camera is taking photos, in order to identify the position and size of acne and blackheads deep in the skin, which helps to improve the recognition rate of acne and blackheads. The set of red LED lights in the range of 630 to 640 nm is used for beauty such as skin rejuvenation.

Preferably, the suction nozzle assembly 5 comprises a nozzle body 51, and a nozzle tip 52 capped on the nozzle body 51. A light-transmitting lens 10 and a sealing ring 20 are disposed between the nozzle body 51 and the nozzle tip 52. Further, the nozzle tip 52 has a silicone portion disposed at the suction opening thereof. The silicone portion is formed of soft silicone and covered around the suction opening of the nozzle tip 52 to achieve soft skin-friendly contact with the skin when sucking blackheads and acne, helping to improve the user experience. The sealing ring 20 plays a role in sealing and fixing.

Preferably, the suction control module 3 comprises a vacuum pump 31 and a motor 32 that controls the vacuum pump 31. The vacuum pump 31 is provided with an air inlet hole and an air outlet hole. The air outlet hole communicates with the external space. The air inlet hole communicates with the nozzle body 51 through a tube. The motor 32 is powered by 5V and has a maximum speed of 6800 RPM.

Preferably, the visual beauty instrument further comprises a power module 7. The power module 7 comprises a battery 71, and a battery management device 72 electrically connected with the battery 71. The battery management device 72 is respectively electrically connected to the control module 1, the image acquisition module 2, the suction control module 3, the fill light module 4 and the mobile terminal 6. The power module 7 supports simultaneous charging and discharging of the battery. The battery 71 uses 18650 2000 mAh polymer battery and has a charging current of 1000 mA. It can charge the mobile terminal 6 through data transmission. The battery management device 72 is an ETA6085 chip, which is a switching power supply chip with high conversion efficiency and low heat generation.

Preferably, the visual beauty instrument further comprises a housing 30. The housing 30 is provided with a control button set 8, which includes a power button 81 and a mode switching button 82. The power button 81 and the mode switching button 82 are respectively electrically connected to the control module 1. The user can press the control button set 8 to switch the working mode and adjust the suction power. The power button 81 integrates on/off and gear shifting. The mode switching button 82 can open the image display module 62 and drive the image acquisition module 2 to capture images. When the image display module 62 is turned off, the control module 1 turns on the set of blue LED lights of 410 to 420 nm wavelength to treat acne and acne anti-inflammatory.

Preferably, the housing 30 is further provided with an indicator light set 9. The indicator light set 9 includes a plurality of suction force level indicator lights 91 and a power status indicator light 92. The suction force level indicator lights 91 and the power status indicator light 92 are electrically connected to the control module 1. The suction force level indicator lights 91 are convenient for the user to know the level of the suction force of the beauty instrument in time.

The working principle of the present invention is:

The image acquisition module 2 is a fixed-focus macro camera with multiple image enlargement function and digital zoom function. It is used to capture the image or video of the skin in the suction area and send the captured image or video to the control module 1 for analyzing the distribution and size of acne and blackheads in the inner layer. Then, the control module 1 controls the suction control module 3 to output the negative pressure of different levels to adjust the suction, avoiding redness or damage to the skin due to excessive suction. At the same time, during image acquisition, the control module 1 controls the fill light module 4 to fill light, improving image quality the recognition of acne and blackheads. When the suction nozzle assembly 5 is in contact with the skin, the vacuum pressure generated by the suction control module 3 sucks and removes the acne and blackheads deep in the skin.

The image analysis module 61 and the image display module 62 are integrated on the app of mobile terminal 6 and communicate with the control module 1. Through the touch screen, you can realize the functions of taking photos and videos, browsing and playing functions of photos and video files, sharing functions of related files, copying, pasting and deleting functions. Further, through instant two-way communication with the control module 1, you can display the battery power of the visual beauty instrument on the touch screen of the mobile terminal 6.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A visual beauty instrument, comprising a control module, an image acquisition module, an suction control module, a fill light module, a suction nozzle assembly and a mobile terminal, said image acquisition module being adapted for collecting an image signal of a suction area on the skin of a person to be sucked by said suction nozzle assembly and feeding back said image signal to said control module, said control module being adapted for analyzing said image signal and control said suction control module and said fill light module, said suction control module being adapted for sucking up the acne and blackheads on the skin surface by said suction nozzle assembly, said fill light module being adapted for improving the recognition of acne and blackheads, said mobile terminal and said control module being communicatively connected, said mobile terminal being integrated with an image analysis module and an image display module, said image analysis module being adapted for analyzing image information, said image display module being adapted for converting image information into images or videos.

2. The visual beauty instrument as claimed in claim 1, wherein said suction control module comprises a vacuum pump and a motor that controls said vacuum pump, said vacuum pump comprising an air inlet hole and an air outlet hole, said air outlet hole communicating with the external space; said suction nozzle assembly comprises a nozzle body, a nozzle tip capped on said nozzle body and a light-transmitting lens and a sealing ring disposed between said nozzle body and said nozzle tip, said nozzle tip having a silicone portion disposed at a suction opening thereof, said nozzle body communicating with said air inlet hole by a tube.

3. The visual beauty instrument as claimed in claim 1, further comprising a housing, a control button set mounted on said housing and an indicator light set mounted on said housing, said control button set comprising a power button and a mode switching button, said power button and said mode switching button being respectively electrically connected to said control module, said indicator light set comprising a plurality of suction force level indicator lights and a power status indicator light, said suction force level indicator lights and said power status indicator light being electrically connected to said control module.

4. The visual beauty instrument as claimed in claim 1, wherein said fill light module comprises at least one set of white LED lights, one set of UVA LED lights having a wavelength in the range of 360 to 400 nm, one set of blue LED lights having a wavelength in the range of 410 to 420 nm, and one set of red LED lights having a wavelength in the range of 630 to 640 nm.
